(19) United States
(12) Patent Application Publication
(10) Pub. No.: US 2024/0116913 A1
(43) Pub. Date: Apr. 11, 2024

(54) A CRYSTALLINE FORM OF (4-METHYL-2-[1,2,3]TRIAZOL-2-YL-PHENYL)-[(R)-3-(3-[1,2,3]TRIAZOL-2-YL-BENZYL)-MORPHOLIN-4-YL]-METHANONE

(71) Applicant: Idorsia Pharmaceuticals Ltd, Allschwil (CH)

(72) Inventor: Markus VON RAUMER, Allschwil (CH)

(21) Appl. No.: 18/263,847

(22) PCT Filed: Jan. 28, 2022
(86) PCT No.: PCT/EP2022/051992

§ 371 Date: Aug. 1, 2023

(30) Foreign Application Priority Data
Feb. 2, 2021 (WO) PCT/EP2021/052373

(51) Int. Cl. C07D 413/14; A61K 9/20; A61K 31/5377

(52) U.S. Cl. CPC C07D 413/14; A61K 9/2013; A61K 9/2018; A61K 9/2027; A61K 9/2054; A61K 9/2095; A61K 31/5377; C07B 2200/13

(57) ABSTRACT

The present invention relates to a crystalline form of (4-Methyl-2-[1,2,3]triazol-2-yl-phenyl)-[(R)-3-(3-[1,2,3]triazol-2-yl-benzyl)-morpholin-4-yl]-methanone, a process for the preparation thereof, pharmaceutical compositions comprising the same and its use as an orexin receptor antagonist in the prevention and/or treatment of various orexin receptor-mediated disorders such as Binge-Eating Disorder (BED).

The present invention relates to a novel crystalline form of the active ingredient (4-Methyl-2-[1,2,3]triazol-2-yl-phenyl)-[(R)-3-(3-[1,2,3]triazol-2-yl-benzyl)-morpholin-4-yl]-methanone (the active ingredient is also referred herein to as ACT-539313), processes for the preparation thereof, pharmaceutical compositions comprising said crystalline form, and its use as orexin receptor antagonist in the prevention and/or treatment of various orexin receptor-mediated diseases, disorders and/or conditions such as anxiety disorders, addiction disorders and eating disorders.

### BACKGROUND ART

ACT-539313 (CAS RN 1435480-40-2) and its orexin receptor antagonistic activity have been previously disclosed in WO2013068935. ACT-539313 is a potent and selective orexin-1 receptor antagonist that has been tested in clinical trials (NCT02702648, NCT03363984, and NCT04753164). Certain clinical aspects of the compound have been discussed by Kaufmann P, et al. Br J Clin Pharmacol. 2020; 86(7):1377-1386; Berger, B, et al. J Clin Pharmacol. 2020; 60(7):931-941; and Kaufmann P, et al. Prog Neuropsychopharmacol Biol Psychiatry. 2021; 108:110166.

### SUMMARY OF INVENTION

The crystalline form of ACT-539313 as disclosed herein may exhibit advantageous physical properties such as being non-hygroscopic and thereby having a longer shelf-life; lower tendency towards agglomeration during storage; simpler packaging/handling; and limited mass variation during weighing/dispensing. The crystalline form may be thermodynamically stable when compared to other thermodynamically less stable or metastable forms. It may have advantageous bulk properties such as powder density and flowability. The polymorphic form disclosed herein may have advantageous powder flow behavior and favourable compression characteristics such as sufficiently high melting point, making it suitable for production of solid dosage forms such as tablets. Further, certain pharmaceutical compositions comprising the crystalline form of the present invention may have advantageous properties such as being particularly suitable for the preparation of both capsules and tablets. Also, the crystalline form may be readily formed in certain solvents as compared to others, where crystallization may take substantially longer.

### BRIEF DESCRIPTION OF DRAWINGS

FIG. 1 shows the X-ray powder diffraction diagram of ACT-539313 in the crystalline form 1, wherein the X-ray powder diffraction diagram is displayed against Cu Kc radiation. The X-ray diffraction diagram shows peaks having a relative intensity, as compared to the most intense peak in the diagram, of the following percentages (relative peak intensities given in parenthesis) at the indicated angles of refraction 2theta (selected peaks from the range 5-35° 2theta with relative intensity larger or equal than 10% are reported): 7.8° (35%), 10.5° (37%), 12.5° (13%), 13.7° (22%), 14.2° (59%), 14.6° (17%), 15.6° (15%), 15.8° (13%), 18.4° (100%), 21.4° (18%), 21.8° (60%), 23.8° (19%), 24.1° (17%), 24.8° (14%), 25.1° (43%), and 25.5° (18%).

In the X-ray diffraction diagrams of FIG. 1 the angle of refraction 2theta (2θ) is plotted on the horizontal axis and the counts on the vertical axis. For avoidance of any doubt, the above-listed peaks describe the experimental results of the X-ray powder diffraction shown in FIG. 1. It is understood that, in contrast to the above peak list, only a selection of characteristic peaks is required to fully and unambiguously characterize ACT-539313 in the respective crystalline form of the present invention.

FIG. 2 shows the X-ray powder diffraction diagram of ACT-539313 in the amorphous state, wherein the X-ray powder diffraction diagram was measured with XRPD method 1 and is displayed against Cu Kα radiation. In the diagram the angle of refraction 20 is plotted on the horizontal axis and the counts on the vertical axis.

FIG. 3 shows the differential scanning calorimetry thermogram of ACT-539313 in the crystalline form 1. Temperature [° C.] is displayed on the x-axis. Heat flow [mW] (endo down) is shown on the y-axis.

FIG. 4 shows the gravimetric vapor sorption isotherm at 25° C. of ACT-539313 in the crystalline form 1. Relative humidity in [%] (25° C.) is displayed on the x-axis. On the y-axis the mass change in [% dry basis] is displayed.

FIG. 5 shows the gravimetric vapor sorption isotherm at 25° C. of ACT-539313 in the amorphous state. Relative humidity in [%] (25° C.) is displayed on the x-axis. On the y-axis the mass change in [% dry basis] is displayed.

### DESCRIPTION OF EMBODIMENTS

1) A first embodiment of the invention relates to a crystalline form of (4-Methyl-2-[1,2,3]triazol-2-yl-phenyl)-[(R)-3-(3-[1,2,3]triazol-2-yl-benzyl)-morpholin-4-yl]-methanone (ACT-539313), characterized by the presence of peaks in the X-ray powder diffraction diagram at the following angles of refraction 2θ: 10.5°, 14.2°, and 18.4°.

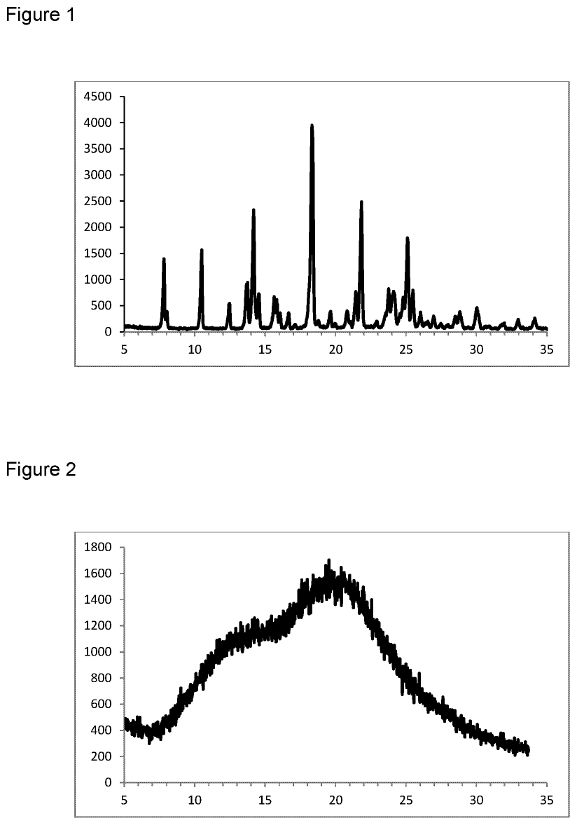

It is understood that the crystalline form according to embodiment 1) comprise ACT-539313 in a crystalline form of the free base (i.e. not in form of a salt). Furthermore, said crystalline form may comprise non-coordinated and/or coordinated solvent. Coordinated solvent is used herein as term for a crystalline solvate. Likewise, non-coordinated solvent is used herein as term for physiosorbed or physically entrapped solvent (definitions according to Polymorphism in the Pharmaceutical Industry (Ed. R. Hilfiker, V C H, 2006), Chapter 8: U. J. Griesser: The Importance of Solvates). The crystalline form of ACT-539313 as disclosed herein comprises no coordinated water but may comprise non-coordinated water or another non-coordinated solvent.
2) Another embodiment relates to the crystalline form of ACT-539313 according to embodiment 1), characterized by the presence of peaks in the X-ray powder diffraction diagram at the following angles of refraction 2θ: 7.8°, 10.5°, 14.2°, 18.4°, and 21.8°.
3) Another embodiment relates to the crystalline form of ACT-539313 according to embodiment 1), characterized by the presence of peaks in the X-ray powder diffraction diagram at the following angles of refraction 2θ: 7.8°, 10.50, 12.50, 13.70, 14.20, 14.60, 18.40, 21.40, 21.80, and 25.10.
4) Another embodiment relates to the crystalline form of ACT-539313 according to embodiment 1), which essentially shows the X-ray powder diffraction pattern as depicted in FIG. 1.
5) Another embodiment relates to the crystalline form of ACT-539313 according to any one of embodiments 1) to 4), characterized by a melting point of about 117.6° C. as determined by differential scanning calorimetry (DSC) using the method as described herein.

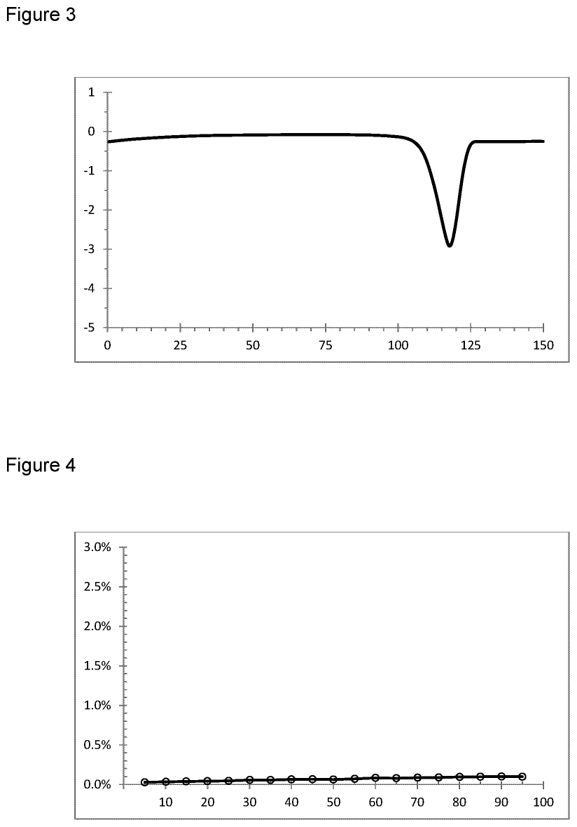

Another embodiment relates to a crystalline form of ACT-539313, characterized by a melting point of 117.6±2° C. ° C. as determined by differential scanning calorimetry (DSC) using the method as described herein.
6) Another embodiment relates to the crystalline form of ACT-539313 according to any one of embodiments 1) to 5), which essentially shows the differential scanning calorimetry profile depicted in FIG. 3.
7) Another embodiment relates to the crystalline form of ACT-539313 according to any one of embodiments 1) to 6), obtainable by the process comprising
   a. dissolving ACT-539313 in a solvent (notably at a concentration of about 1 g of ACT-539313 in from about 1 mL to about 20 mL of solvent), said solvent comprising one or more lower alcohols (notably selected from 1-propanol, 2-propanol, ethanol, or a mixture thereof; especially 2-propanol);
   b. awaiting (notably for less than about 24 h) the formation of a solid product; and
   c. isolating the solid product.

The isolation step as defined herein may be performed by any method known in the art to separate a solid precipitate from a liquid, preferably by filtration.

The term “lower alcohols” as used herein refers to mono-, di- or poly-valent (notably mono- or di-valent; especially mono-valent) alcohols i.e. to alcohols bearing 1, 2, or more hydroxyl groups (notably 1 or 2; especially 1 hydroxyl group), said hydroxyl group(s) being attached to a C₁₋₅-alkane by substitution of one or more hydrogen atoms.

The term “C₁₋₅-alkane” refers to a saturated, straight or branched hydrocarbon chain consisting of one to five carbon atoms. Examples of lower alcohols as used herein are methanol, ethanol, 1-propanol, 2-propanol, 1-butanol, isobutanol, 2-methyl-propan-2-ol, 2-methyl-propan-1-ol, 1-pentanol, 2-pentanol, 3-pentanol, ethylene glycol, propylene glycol, glycerol; notably methanol, ethanol, 1-propanol, 2-propanol; especially ethanol, 1-propanol, and 2-propanol. The solvent used to dissolve ACT-539313 as defined in embodiment 7) or in any of the embodiments disclosed herein comprises at least 50% (notably at least 75%; especially essentially 100%) of said one or more lower alcohols.
8) Another embodiment relates to a process of making the crystalline form of ACT-539313 according to any one of embodiments 1) to 6), said process comprising recrystallization of ACT-539313 in a solvent (notably at a concentration of about 1 g of ACT-539313 in from about 1 mL to about 20 mL of solvent), said solvent comprising one or more lower alcohols (notably selected from 1-propanol, 2-propanol, ethanol, or a mixture thereof; especially 2-propanol); especially said process comprising
   a. dissolving ACT-539313 in a solvent (notably at a concentration of about 1 g of ACT-539313 in from about 1 mL to about 20 mL of solvent), said solvent comprising one or more lower alcohols (notably 1-propanol, 2-propanol, ethanol; especially 2-propanol);
   b. awaiting (notably for less than about 24 h) the formation of a solid product; and
   c. isolating the solid product.

For avoidance of any doubt, whenever one of the above embodiments refers to “peaks in the X-ray powder diffraction diagram at the following angles of refraction 20”, said X-ray powder diffraction diagram is obtained by using combined Cu Kα1 and Kα2 radiation, without Kα2 stripping; and it should be understood that the accuracy of the 2θ values as provided herein is in the range of +/−0.1-0.2°. Notably, when specifying an angle of refraction 2theta (2θ) for a peak in the invention embodiments and the claims, the 2θ value given is to be understood as an interval from said value minus 0.2° to said value plus 0.2° (2θ+/−0.2°); and preferably from said value minus 0.1° to said value plus 0.1° (2θ+/−0.1°).

Where the plural form is used for compounds, solids, pharmaceutical compositions, diseases and the like, this is intended to mean also a single compound, solid, pharmaceutical composition, disease or the like.

Definitions provided herein are intended to apply uniformly to the subject matter as defined in any one of embodiments 1) to 8), and, mutatis mutandis, throughout the description and the claims unless an otherwise expressly set out definition provides a broader or narrower definition. It is well understood that a definition or preferred definition of a term or expression defines and may replace the respective term or expression independently of (and in combination with) any definition or preferred definition of any or all other terms or expressions as defined herein.

When defining the presence of peak in e.g. an X-ray powder diffraction diagram, a common approach is to do this in terms of the S/N ratio (S=signal, N=noise). According to this definition, when stating that a peak has to be present in a X-ray powder diffraction diagram, it is understood that the peak in the X-ray powder diffraction diagram is defined by having an S/N ratio (S=signal, N=noise) of greater than x (x being a numerical value greater than 1), usually greater than 2, especially greater than 3.

In the context of the present invention, when stating that the crystalline form essentially shows an X-ray powder diffraction pattern as depicted in FIG. 1, the term “essentially” means that at least the major peaks of the diagram depicted in said figures, i.e. those having a relative intensity of more than 20%, especially more than 10%, as compared to the most intense peak in the diagram, have to be present. However, the person skilled in the art of X-ray powder diffraction will recognize that relative intensities in X-ray powder diffraction diagrams may be subject to strong intensity variations due to preferred orientation effects.

Unless used regarding temperatures, the term “about” placed before a numerical value “X” refers in the current application to an interval extending from X minus 10% of X to X plus 10% of X, and preferably to an interval extending from X minus 5% of X to X plus 5% of X; most preferred is X. In the particular case of temperatures, the term “about” placed before a temperature “Y” refers in the current application to an interval extending from the temperature Y minus 10° C. to Y plus 10° C., preferably to an interval extending from Y minus 5° C. to Y plus 5° C. Room temperature means a temperature of about 25° C.

Whenever the word “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

The crystalline form of ACT-539313 according to any one of embodiments 1) to 7) can be used as a medicament, e.g. in the form of pharmaceutical compositions for enteral (such as especially oral) administration.
9) Another embodiment thus relates to a crystalline form of ACT-539313 according to any one of embodiments 1) to 7) for use as a medicament.

The crystalline solid of ACT-539313 according to any one of embodiments 1) to 7) may be used as single component or as mixture with other crystalline forms or amorphous form of ACT-539313.
10) A further embodiment relates to pharmaceutical compositions comprising as active ingredient a crystalline form of ACT-539313 according to any one of embodiments 1) to 7), and at least one pharmaceutically acceptable carrier material. Notably, the composition is a solid pharmaceutical composition, notably for oral use, especially a tablet (for oral use).

In a preferred embodiment the crystalline form of ACT-539313 according to any one of embodiments 1) to 7) is comprised in a solid dosage form (for oral use). Notably, the solid dosage form is a tablet, capsule, sphere, granulate, lyophilizate, thin film; notably the solid dosage form is a tablet or capsule (for oral use); especially tablet (for oral use).

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the crystalline form of the present invention, optionally in combination with other therapeutically valuable substances, into a galenical administration form together with suitable, non-toxic, inert, pharmaceutically acceptable solid or liquid carrier materials and, if desired, usual pharmaceutical adjuvants.
11) A further embodiment relates to a pharmaceutical composition (notably a tablet or capsule for oral use; especially a tablet for oral use) according to embodiment 10) comprising
   from about 3% w/w to about 40% w/w of said active ingredient;
   from about 30% w/w to about 70% w/w microcrystalline cellulose;
   from about 15% w/w to about 60% w/w mannitol;
   from about 3% w/w to about 10% w/w crosspovidone;
   from about 0.2% w/w to about 4% w/w sodium stearyl fumarate; and
   from about 0.5% w/w to about 5% w/w silica colloidal hydrated;
[wherein the sum of the components of said composition (notably tablet or capsule; especially tablet) is 100%];
or notably comprising
   from about 4% w/w to about 35% w/w of said active ingredient;
   from about 35% w/w to about 60% w/w microcrystalline cellulose;
   from about 20% w/w to about 40% w/w mannitol;
   from about 3% w/w to about 8% w/w crosspovidone;
   from about 0.5% w/w to about 3% w/w sodium stearyl fumarate; and
   from about 1% w/w to about 3% w/w silica colloidal hydrated;
[wherein the sum of the components of said composition (especially tablet) is 100%];
or especially comprising
   from about 5% w/w to about 25% w/w of said active ingredient;
   from about 38% w/w to about 55% w/w microcrystalline cellulose;
   from about 24% w/w to about 35% w/w mannitol;
   from about 4% w/w to about 6% w/w crosspovidone;
   from about 0.5% w/w to about 2% w/w sodium stearyl fumarate; and
   from about 1% w/w to about 2.5% w/w silica colloidal hydrated;
[wherein the sum of the components of said composition (especially tablet) is 100%];
12) A further embodiment relates to a pharmaceutical composition (notably a tablet or capsule for oral use; especially a tablet for oral use) according to embodiment 10) comprising
from 22% to 25% w/w of said active ingredient;
   from 39% to 44% w/w microcrystalline cellulose;
   from 24% to 30% w/w mannitol;
   from 4% to 6% w/w crosspovidone;
   from 0.5% to 1.5% w/w sodium stearyl fumarate; and
   from 1.5% to 2.5% w/w silica colloidal hydrated;
[wherein the sum of the components of said composition (notably tablet or capsule; especially tablet) is 100%];
or
   from 10% to 13% w/w of said active ingredient;
   from 47% to 52% w/w microcrystalline cellulose;
   from 29% to 34% w/w mannitol;
   from 4% to 6% w/w crosspovidone;
   from 0.5% to 1.5% w/w sodium stearyl fumarate; and
   from 1% to 2% w/w silica colloidal hydrated;
[wherein the sum of the components of said composition (notably tablet or capsule; especially tablet) is 100%];
or
   from 6% to 8% w/w of said active ingredient;
   from 48% to 54% w/w microcrystalline cellulose;
   from 31% to 35% w/w mannitol;
   from 4% to 6% w/w crosspovidone;
   from 0.5% to 1.5% w/w sodium stearyl fumarate; and
   from 1% to 2% w/w silica colloidal hydrated;
[wherein the sum of the components of said composition (notably tablet or capsule; especially tablet) is 100%];
or
   from 5% to 7% w/w of said active ingredient;
   from 50% to 55% w/w microcrystalline cellulose;
   from 30% to 35% w/w mannitol;
   from 4% to 6% w/w crosspovidone;
   from 0.5% to 1.5% w/w sodium stearyl fumarate; and
   from 0.5% to 2% w/w silica colloidal hydrated;
[wherein the sum of the components of said composition (notably tablet or capsule; especially tablet) is 100%];

It is understood that the term “components” as used herein refers to the active ingredient, the respective excipients (e.g. microcrystalline cellulose, mannitol, etc.), and further components (e.g. small amounts of impurities, etc.) that may be present but do not materially affect the essential characteristics of the corresponding pharmaceutical compositions. The term “comprising” as used in embodiments 11) and 12) and in the respective claims may be replaced by the term “consisting essentially of”, as expedient and appropriate.
13) A further embodiment relates to a crystalline form of ACT-539313 according to any one of embodiments 1) to 7), for use in the manufacture of a (solid) pharmaceutical composition, wherein said pharmaceutical composition comprises as active ingredient ACT-539313, and at least one pharmaceutically acceptable carrier material.
14) A further embodiment of the invention relates to a crystalline form of ACT-539313 according to any one of embodiments 1) to 7), or pharmaceutical compositions (especially a tablet for oral use) according to any one of embodiments 10) to 12), for use in the prevention/prophylaxis and/or treatment of a disease or disorder associated with an orexinergic dysfunction; and notably of a disease or disorder, wherein the blockade of at least one orexin receptor (especially the blockade of the OX1 receptor) is indicated.

Disorders relating to orexinergic dysfunctions are diseases or disorders where an antagonist of a human orexin receptor is required, notably mental health diseases or disorders relating to orexinergic dysfunctions, notably of the OX1 receptor. The above-mentioned disorders may in particular be defined as comprising anxiety disorders, addiction disorders, mood disorders, or appetite disorders, as well as cognitive dysfunctions or sleep disorders. Especially, the above-mentioned disorders comprise anxiety disorders, addiction disorders and mood disorders, notably anxiety disorders and addiction disorders.

Anxiety disorders can be distinguished by the primary object or specificity of threat, ranging from rather diffuse as in generalized anxiety disorder, to circumscribed as encountered in phobic anxieties (PHOBs) or post-traumatic stress disorders (PTSDs). Anxiety disorders may, thus, be defined as comprising generalized anxiety disorders (GAD), obsessive compulsive disorders (OCDs), acute stress disorders, posttraumatic stress disorders (PTSDs), panic anxiety disorders (PADs) including panic attacks, phobic anxieties (PHOBs), specific phobia, social phobia (social anxiety disorder), avoidance, somatoform disorders including hypochondriasis, separation anxiety disorder, anxiety disorders due to a general medical condition, and substance induced anxiety disorders. In a sub-embodiment, particular examples of circumscribed threat induced anxiety disorders are phobic anxieties or post-traumatic stress disorders. Anxiety disorders especially include generalized anxiety disorders, post-traumatic stress disorders, obsessive compulsive disorders, panic attacks, phobic anxieties, and avoidance.

Addiction disorders may be defined as addictions to one or more rewarding stimuli, notably to one rewarding stimulus. Such rewarding stimuli may be of either natural or synthetic origin. Examples of such rewarding stimuli are substances/drugs {of either natural or synthetic origin; such as cocaine, amphetamines, opiates [of natural or (semi-)synthetic origin such as morphine or heroin], cannabis, ethanol, mescaline, nicotine, and the like}, which substances/drugs may be consumed alone or in combination; or other rewarding stimuli {of either natural origin (such as food, sweet, fat, or sex, and the like), or synthetic origin [such as gambling, or internet/IT (such as immoderate gaming, or inappropriate involvement in online social networking sites or blogging), and the like]}. In a sub-embodiment, addiction disorders relating to psychoactive substance use, abuse, seeking and reinstatement are defined as all types of psychological or physical addictions and their related tolerance and dependence components. Substance-related addiction disorders especially include substance use disorders such as substance dependence, substance craving and substance abuse; substance-induced disorders such as substance intoxication, substance withdrawal, and substance-induced delirium. The expression “prevention or treatment of addictions” (i.e. preventive or curative treatment of patients who have been diagnosed as having an addiction, or as being at risk of developing addictions) refers to diminishing addictions, notably diminishing the onset of addictions, to weakening their maintenance, to facilitating withdrawal, to facilitating abstinence, or to attenuating, decreasing or preventing the occurrence of reinstatement of addiction (especially to diminishing the onset of addictions, to facilitating withdrawal, or to attenuating, decreasing or preventing the occurrence of reinstatement of addiction).

Mood disorders include major depressive episode, manic episode, mixed episode and hypomanic episode; depressive disorders including major depressive disorder, dysthymic disorders; bipolar disorders including bipolar I disorder, bipolar II disorder (recurrent major depressive episodes with hypomanic episodes), cyclothymic disorder; mood disorders including mood disorder due to a general medical condition (including the subtypes with depressive features, with major depressive-like episode, with manic features, and with mixed features), substance-induced mood disorder (including the subtypes with depressive features, with manic features, and with mixed features). Such mood disorders are especially major depressive episode, major depressive disorder, mood disorder due to a general medical condition; and substance-induced mood disorder.

Appetite disorders comprise eating disorders and drinking disorders. Eating disorders may be defined as comprising eating disorders associated with excessive food intake and complications associated therewith; anorexias; compulsive eating disorders; obesity (due to any cause, whether genetic or environmental); obesity-related disorders including overeating and obesity observed in Type 2 (non-insulin-dependent) diabetes patients; bulimias including bulimia nervosa; cachexia; and most notably binge eating disorder or bulimia nervosa. Particular eating disorders comprise metabolic dysfunction; dysregulated appetite control; compulsive obesities; bulimia or anorexia nervosa. In a sub-embodiment, eating disorders may be defined as especially comprising anorexia nervosa, bulimia nervosa, cachexia, binge eating disorder, or compulsive obesities, most preferably binge eating disorder and/or bulimia nervosa. Drinking disorders include polydipsias in psychiatric disorders and all other types of excessive fluid intake. Pathologically modified food intake may result from disturbed appetite (attraction or aversion for food); altered energy balance (intake vs. expenditure); disturbed perception of food quality (high fat or carbohydrates, high palatability); disturbed food availability (unrestricted diet or deprivation) or disrupted water balance.

Cognitive dysfunctions include deficits in attention, learning and especially memory functions occurring transiently or chronically in psychiatric, neurologic, neurodegenerative, cardiovascular and immune disorders, and also occurring transiently or chronically in the normal, healthy, young, adult, or especially aging population. Cognitive dysfunctions especially relate to the enhancement or maintenance of memory in patients who have been diagnosed as having, or being at risk of developing, diseases or disorders in which diminished memory (notably declarative or procedural) is a symptom [in particular dementias such as frontotemporal dementia, or dementia with Lewy bodies, or (especially) Alzheimer's disease]. Especially, the term “prevention or treatment of cognitive dysfunctions” relates to the enhancement or maintenance of memory in patients who have a clinical manifestation of a cognitive dysfunction, especially expressed as a deficit of declarative memory, linked to dementias such as frontotemporal dementia, or dementia with Lewy bodies, or (especially) Alzheimer's disease. Furthermore, the term “prevention or treatment of cognitive dysfunctions” also relates to improving memory consolidation in any of the above-mentioned patient populations.

Sleep disorders comprise dyssomnias, parasomnias, sleep disorders associated with a general medical condition and substance-induced sleep disorders. In particular, dyssomnias include intrinsic sleep disorders (especially insomnias, breathing-related sleep disorders, periodic limb movement disorder, and restless leg syndrome), extrinsic sleep disorders, and circadian-rhythm sleep disorders. Dyssomnias notably include insomnia, primary insomnia, idiopathic insomnia, insomnias associated with depression, emotional/mood disorders, aging, Alzheimer's disease or cognitive impairment; REM sleep interruptions; breathing-related sleep disorders; sleep apnea; periodic limb movement disorder (nocturnal myoclonus), restless leg syndrome, circadian rhythm sleep disorder; shift work sleep disorder; and jet-lag syndrome. Parasomnias include arousal disorders and sleep-wake transition disorders; notably parasomnias include nightmare disorder, sleep terror disorder, and sleepwalking disorder. Sleep disorders associated with a general medical condition are in particular sleep disorders associated with diseases such as mental disorders, neurological disorders, neuropathic pain, and heart and lung diseases. Substance-induced sleep disorders include especially the subtypes insomnia type, parasomnia type and mixed type, and notably include conditions due to drugs which cause reductions in REM sleep as a side effect. Sleep disorders especially include all types of insomnias, sleep-related dystonias; restless leg syndrome; sleep apneas; jet-lag syndrome; shift work sleep disorder, delayed or advanced sleep phase syndrome, or insomnias related to psychiatric disorders. In addition, sleep disorders further include sleep disorders associated with aging; intermittent treatment of chronic insomnia; situational transient insomnia (new environment, noise) or short-term insomnia due to stress; grief; pain or illness.
15) A further embodiment relates to a crystalline form of ACT-539313 according to any one of embodiments 1) to 7), or pharmaceutical compositions (especially a tablet for oral use) according to any one of embodiments 10) to 12), for use in the prevention/prophylaxis and/or treatment of a disease or disorder selected from addiction disorders, anxiety disorders, appetite disorders, cognitive dysfunctions and mood disorders (notably addiction disorders, anxiety disorders and appetite disorders; especially appetite disorders; in particular binge eating disorder).
16) A further embodiment relates to a crystalline form of ACT-539313 according to any one of embodiments 1) to 7), or pharmaceutical compositions (especially a tablet for oral use) according to any one of embodiments 10) to 12), for use in the prevention/prophylaxis and/or treatment of eating disorders, wherein the eating disorder is an eating disorder comprising a compulsive, binge eating behavior.

It is understood that the term “eating disorder comprising a compulsive, binge eating behavior” refers to a disorder comprising recurring episodes of binge eating, i.e. recurring episodes when a subject is eating significantly more food in a short period of time than most people would eat under similar circumstances, with episodes marked by feelings of lack of control. Eating disorder comprising a compulsive, binge eating behavior is characterized by eating large amounts of food, by eating quickly (often to the point of discomfort), and by eating even when no longer hungry.
17) A further embodiment relates to a crystalline form of ACT-539313 according to any one of embodiments 1) to 7), or pharmaceutical compositions (especially a tablet for oral use) according to any one of embodiments 10) to 12), for use in the prevention/prophylaxis and/or treatment of eating disorders, wherein the eating disorder is selected from a group comprising Binge-Eating Disorder (BED); Bulimia Nervosa (BN); Anorexia Nervosa (AN) (notably binge-eating/purging type Anorexia Nervosa; especially binge-eating type Anorexia Nervosa); Pica; Other Specified Feeding and Eating Disorders (OSFED) [notably atypical Bulimia Nervosa, Binge-Eating Disorder of low frequency and/or limited duration, Bulimia Nervosa of low frequency and/or limited duration, or Night Eating Syndrome (NES)]; Unspecified Feeding or Eating Disorder (UFED); Eating Disorder Not Otherwise Specified (EDNOS); and Compulsive Overeating (CO); The eating disorder is further selected from a group comprising Loss of Control (LOC) Eating; and especially hyperphagia and/or binge-eating, associated with Prader-Willi Syndrome (PWS).
18) A further embodiment relates to a crystalline form of ACT-539313 according to any one of embodiments 1) to 7), or pharmaceutical compositions (especially a tablet for oral use) according to any one of embodiments 10) to 12), for use in the prevention/prophylaxis and/or treatment of eating disorders, wherein the eating disorder is Binge-Eating Disorder (BED), Bulimia Nervosa (BN), or binge-eating type Anorexia Nervosa. Especially, Binge-Eating Disorder (BED).

The American Psychiatric Association's Diagnostic and Statistical Manual of Mental Disorders (DSM-5® or herein also referred to as DSM-5) provides diagnostic criteria for certain feeding and eating disorders.

Binge-Eating Disorder (BED) is defined as recurring episodes of eating significantly more food in a short period of time than most people would eat under similar circumstances, with episodes marked by feelings of lack of control. Someone with binge eating disorder may eat too quickly, even when they are not hungry. The person may have feelings of guilt, embarrassment, or disgust and may binge eat alone to hide the behaviour. BED is associated with marked distress and significant physical, emotional, and social health risks such as obesity and extreme weight gain and a wide range of associated diseases such as sleep apnea, cancer, heart disease, high blood pressure, type 2 diabetes, arthritis, etc., being among the most common ones.

According to DSM-5®, 307.51 (F50.8), the following diagnostic criteria for Binge-Eating Disorder are provided:
A. Recurrent episodes of binge eating. An episode of binge eating is characterized by both of the following: 1. Eating, in a discrete period of time (e.g., within any 2-hour period), an amount of food that is definitely larger than what most people would eat in a similar period of time under similar circumstances; 2. A sense of lack of control overeating during the episode (e.g., a feeling that one cannot stop eating or control what or how much one is eating).
B. The binge-eating episodes are associated with three (or more) of the following: 1. Eating much more rapidly than normal; 2. Eating until feeling uncomfortably full; 3. Eating large amounts of food when not feeling physically hungry; 4. Eating alone because of feeling embarrassed by how much one is eating; 5. Feeling disgusted with oneself, depressed, or very guilty afterward; C. Marked distress regarding binge eating is present; D. The binge eating occurs, on average, at least once a week for 3 months.
E. The binge eating is not associated with the recurrent use of inappropriate compensatory behavior as in bulimia nervosa and does not occur exclusively during the course of bulimia nervosa or anorexia nervosa.

In addition, DSM-5® specifies the criterial for full- and partial remission of BED and its severity. Thus, partial BED remission is defined as: After full criteria (A-E) for binge-eating disorder were previously met, binge eating occurs at an average frequency of less than one episode per week for a sustained period of time. In full remission: After full criteria for binge-eating disorder were previously met, none of the criteria have been met for a sustained period of time.

The minimum level of severity of BED is based on the frequency of episodes of binge eating. The following levels of severity are defined: Mild: 1-3 binge-eating episodes per week. Moderate: 4-7 binge-eating episodes per week. Severe: 8-13 binge-eating episodes per week. Extreme: 14 or more binge-eating episodes per week.

Bulimia Nervosa (BN), also known as simply bulimia, is an eating disorder characterized by binge eating followed by self-induced compensatory behavior (purging) to avoid weight gain. Purging may be induced by vomiting or taking laxatives, use of diuretics, stimulants, water fasting, etc. Bulimia is frequently associated with other mental disorders such as depression, anxiety, problems with drugs or alcohol and a higher risk of suicide and self-harm DSM-5® diagnostic criteria sets the frequency of binge eating and compensatory behaviors (purging) that people with bulimia nervosa must exhibit at once a week.

Anorexia Nervosa (AN) primarily affects adolescent girls and young women and is characterized by distorted body image and excessive dieting that leads to severe weight loss with a pathological fear of becoming fat. This condition has potentially life-threatening physiologic effects and causes lasting psychological disturbance.

Diagnostic criteria for AN in the DSM-5 include the following: I) Restriction of energy intake relative to requirements, leading to a significantly low body weight in the context of age, sex, developmental trajectory, and physical health; significantly low weight is defined as a weight that is less than minimally normal or, for children and adolescents, less than that minimally expected; II) Intense fear of gaining weight or of becoming fat, or persistent behavior that interferes with weight gain, even though the patient's weight is already significantly low; III) Disturbance in the way in which one's body weight or shape is experienced, undue influence of body weight or shape on self-evaluation, or persistent lack of recognition of the seriousness of the current low body weight.

A subtype of Anorexia Nervosa (AN) is binge-eating/purging type AN. According to the diagnostic criteria set forth in the Structured Clinical Interview for DSM-5 (SCID-RV (for DSM-5 ®), Version 1.0.0), patients suffering from AN fall within this subtype if they also engage in recurrent episodes of binge-eating or purging behavior (i.e., self-induced vomiting or misuse of laxatives, diuretics, or enemas) for three months.

Other Specified Feeding and Eating Disorders (OSFED) are feeding and eating disorders of clinical severity that do not meet diagnostic criteria for AN, BN, BED, or Pica. They may include atypical AN, atypical BN, BED of low frequency and/or limited duration, and Night Eating Syndrome (NES). Notably, the group of Other Specified Feeding and Eating Disorders (OSFED) as referred herein includes atypical BN; BED of low frequency and/or limited duration; and Night Eating Syndrome (NES).

Unspecified Feeding or Eating Disorder (UFED) exists when an individual's symptoms do not meet criteria for those of another disorder, or when there is simply not enough information to determine a more specific diagnosis. UFED is considered to fall under the Other Specified Feeding or Eating Disorders (OSFED) spectrum, previously known as Eating Disorder Not Otherwise Specified (EDNOS) in past editions of the DSM (older than DSM-5). Despite being considered a ‘catch-all’ classification, OSFED/EDNOS is a serious, life-threatening, and treatable eating disorder. The category was developed to encompass those individuals who did not meet strict diagnostic criteria for AN or BN but still had a significant eating disorder. In community clinics, the majority of individuals were historically diagnosed with EDNOS.

In the DSM-5®, a person with OSFED presents with feeding or eating behaviors that cause clinically significant distress and impairment, but do not meet the full criteria for any of the other disorders. The following are examples of OSFED: Atypical AN: All criteria for AN are met, except despite significant weight loss, the individual's weight is within or above the normal range; BED of low frequency and/or limited duration: All of the criteria for BED are met, except at a lower frequency and/or for less than three months; BN of low frequency and/or limited duration: All of the criteria for BN are met, except that the binge-eating and inappropriate compensatory behavior occurs at a lower frequency and/or for less than three months; Purging Disorder: Recurrent purging behavior to influence weight or shape in the absence of binge eating. Night Eating Syndrome: Recurrent episodes of night eating. Eating after awakening from sleep, or by excessive food consumption after the evening meal. The behavior is not better explained by environmental influences or social norms. The behavior causes significant distress/impairment. The behavior is not better explained by another mental health disorder (e.g. BED).

Compulsive Overeating (CO) (also known as food addiction) is a term commonly referred to for people who identify with an intense urge or compulsion (impulsive behavior) to consume large amounts of food in a relatively short period of time. CO may be used synonymously with BED.

Pica may be present in conjunction with other feeding and eating disorders. DSM-5® criteria for pica are as follows: 1) Persistent eating of non-nutritive, non-food substances over a period of at least 1 month; 2) The eating of such substances is inappropriate to the developmental level of the individual; 3) The eating behavior is not part of a culturally supported or socially normative practice; 4) If the behavior occurs within the context of another mental disorder or medical condition (e.g., schizophrenia, autism, or pregnancy), it is sufficiently severe to warrant independent clinical attention.

Loss of Control (LOC) Eating: A sense of LOC during binge episodes is a core feature of BED. The term “LOC eating” is used to describe these episodes, but is also used more broadly throughout the literature to describe binge-like eating behaviour accompanied by a sense of LOC that occurs across a wide spectrum of individuals. The spectrum includes, among others, individuals who exhibit some features of BED but do not meet full diagnostic criteria for the disorder (i.e. subthreshold BED) and individuals with other eating disorders (bulimia nervosa, anorexia nervosa binge-eating/purge subtype). The spectrum of those described as exhibiting LOC eating also includes individuals for whom diagnosis of threshold BED is challenging for unique reasons, such as post bariatric surgery patients and young children.

Prader-Willi Syndrome (PWS) is a neurodevelopmental disorder genetically determined by a loss of function of specific genes on chromosome 15. The disorder is associated, among others, with hyperphagia leading to severe obesity and other behavioral problems, which may result in a debilitating physical and developmental disability in adolescence and adulthood. Hyperphagia in PWS manifests as an intense persistent sensation of hunger accompanied by food preoccupations, an extreme drive to consume food, food-related behavior problems, and a lack of normal satiety. PWS-associated hyperphagia has overlap with binge eating disorder and obsessive-compulsive features (preoccupation with food); (e.g. Heymsfield S B et al., Obesity. 2014; 22(S1):S1-17.doi.org/10.1002/oby.20646; and Schwartz L et al., J Neurodev Disord. 2021 Jun. 21; 13(1):25. doi: 10.1186/s11689-021-09373-2).

The term “treat” or “treatment” or “treating” used with reference to a disease/disorder/condition means either that said disease/disorder/condition is cured in the subject; or that, although the subject remains affected by the disease, part or all of the symptoms of said disease are either reduced or eliminated.

The term “prevention” as used herein refers to maintenance of efficacy with long-term treatment of the disorders mentioned in the present application, e.g., the term may refer to reduction or prevention of future episodes of binge eating (and related behavior). The term “prevention” as used herein may refer to reduction or prevention of relapse/recurrence of the disorders disclosed herein. Especially, the term refers to prevention of relapse of the disorders (such as eating disorders e.g., BED, BN and binge-eating type Anorexia Nervosa) disclosed herein.

Such reduction or prevention of relapse/recurrence of a disorder is common in the field of psychiatric conditions like depression (e.g. prevention of relapse of depression or recurrence of psychosis) as evident by studies demonstrating that a drug reduces or prevents relapse and maintains efficacy [e.g. Hudson et al., JAMA Psychiatry. 2017 Sep. 1; 74(9):903-910. (PMID: 28700805); Romano et al., Am J Psychiatry. 2002 January; 159(1):96-102. doi: 10.1176/appi.ajp.159.1.96. (PMID: 11772696); and Dobson et al., J Consult Clin Psychol. 2008 June; 76(3):468-77. (PMID: 18540740)]. The term “relapse” is defined as a full return of (binge eating) symptoms once remission has occurred—but before recovery has taken hold. The term “recurrence” refers to another episode of a (binge-eating) behavior after recovery has been attained. The term “prevention” may be understood as being equivalent to the term “prophylaxis”.

The terms “subject(s)”, and likewise, “patient(s)” refers to mammal(s), especially human(s).

For avoidance of any doubt, if a crystalline form of ACT-539313 is described as useful for the prevention/prophylaxis and/or treatment of certain diseases, such crystalline form of ACT-539313 is likewise suitable for use in the preparation of a medicament for the prevention or treatment of said diseases.
19) A further embodiment relates to a method of prevention and/or treatment of a disease/disorder according to any one of embodiments 14) to 18) (especially according to embodiment 18)), said method comprising administering to a subject in need of said prevention and/or treatment an effective amount of the active ingredient (4-methyl-2-[1,2,3]triazol-2-yl-phenyl)-[(R)-3-(3-[1,2,3]triazol-2-yl-benzyl)-morpholin-4-yl]-methanone, or the pharmaceutical compositions (especially a tablet for oral use) according to any one of embodiments 10) to 12); wherein said active ingredient is in the crystalline form according to any one of embodiments 1) to 7).
20) A further embodiment relates to the method of claim 19), wherein the amount is from about 20 mg/day to about 250 mg/day; from about 20 mg/day to about 225 mg/day; from about 20 mg/day to about 200 mg/day; from about 20 mg/day to about 175 mg/day; from about 20 mg/day to about 150 mg/day; from about 20 mg/day to about 125 mg/day; from about 20 mg/day to about 100 mg/day; from about 20 mg/day to about 75 mg/day; from about 20 mg/day to about 80 mg/day; from about 20 mg/day to about 60 mg/day; or from about 20 mg/day to about 50 mg/day. Notably, the amount is from about 20 mg/day to about 150 mg/day; from about 20 mg/day to about 100 mg/day; or from about 20 mg/day to about 80 mg/day. Especially, the amount is from about 20 mg/day to about 200 mg/day.
21) A further embodiment relates to the method according to embodiment 19), wherein the amount is equal to about 30 mg/day; equal to about 35 mg/day; equal to about 40 mg/day; equal to about 45 mg/day; equal to about 50 mg/day; equal to about 55 mg/day; equal to about 60 mg/day; equal to about 65 mg/day; equal to about 70 mg/day; equal to about 75 mg/day; equal to about 80 mg/day; equal to about 100 mg/day; equal to about 125 mg/day; or equal to about 150 mg/day. Especially, the amount is equal to about 60 mg/day.
22) Another aspect of the present invention relates to the active ingredient (4-methyl-2-[1,2,3]triazol-2-yl-phenyl)-[(R)-3-(3-[1,2,3]triazol-2-yl-benzyl)-morpholin-4-yl]-methanone in the crystalline form according to any one of embodiments 1) to 7), or the pharmaceutical compositions (especially a tablet for oral use) according to any one of embodiments 10) to 12); for use in the prevention and/or treatment of a disease/disorder according to any one of embodiments 14) to 18) (especially according to embodiment 18)), wherein the said crystalline form is administered to a subject in the amount according to embodiments 20) and 21).
23) Another aspect of the present invention relates to the use of active ingredient (4-methyl-2-[1,2,3]triazol-2-yl-phenyl)-[(R)-3-(3-[1,2,3]triazol-2-yl-benzyl)-morpholin-4-yl]-methanone in the crystalline form according to any one of embodiments 1) to 7), or the pharmaceutical compositions (especially a tablet for oral use) according to any one of embodiments 10) to 12); (in the preparation of a medicament) for the prevention and/or treatment of a disease/disorder according to any one of embodiments 14) to 18) (especially according to embodiment 18)), wherein the said crystalline form is administered to a subject in the amount according to embodiments 20) and 21).

Another aspect of the present invention relates to the pharmaceutical compositions according to any one of embodiments 9) to 18), wherein the active ingredient is 4-Methyl-2-[1,2,3]triazol-2-yl-phenyl)-[(R)-3-(3-[1,2,3]triazol-2-yl-benzyl)-morpholin-4-yl]-methanone (ACT-539313) [in any crystalline form, amorphous state, or a mixture thereof].

Another embodiment of the present invention relates to a method of prevention and/or treatment of a disease/disorder according to any one of embodiments 14) to 18) (especially according to embodiment 18)), said method comprising administering to a subject in need of said prevention and/or treatment an effective amount of the pharmaceutical compositions (especially a tablet for oral use) according to any one of embodiments 9) to 18); wherein said active ingredient is 4-Methyl-2-[1,2,3]triazol-2-yl-phenyl)-[(R)-3-(3-[1,2,3]triazol-2-yl-benzyl)-morpholin-4-yl]-methanone (ACT-539313) [in any crystalline form, amorphous state, or a mixture thereof].

Yet another embodiment of the present invention relates to the pharmaceutical compositions (especially a tablet for oral use) according to any one of embodiments 9) to 18), for use in the prevention and/or treatment of a disease/disorder according to any one of embodiments 14) to 18) (especially according to embodiment 18)), wherein said active ingredient is 4-Methyl-2-[1,2,3]triazol-2-yl-phenyl)-[(R)-3-(3-[1,2,3]triazol-2-yl-benzyl)-morpholin-4-yl]-methanone (ACT-539313) [in any crystalline form, amorphous state, or a mixture thereof].

Based on the dependencies of the different embodiments 1) to 23) as disclosed hereinabove, the following embodiments are thus possible and intended and herewith specifically disclosed in individualized form: 2+1, 3+1, 4+1, 5+1, 6+1, 6+2+1, 6+3+1, 6+4+1, 6+5+1, 7+1, 7+2+1, 7+3+1, 7+4+1, 7+5+1, 7+6+1, 7+6+2+1, 7+6+3+1, 7+6+4+1, 7+6+5+1, 8+1, 8+2+1, 8+3+1, 8+4+1, 8+5+1, 8+6+1, 8+6+2+1, 8+6+3+1, 8+6+4+1, 8+6+5+1, 9+1, 9+2+1, 9+3+1, 9+4+1, 9+5+1, 9+6+1, 9+6+2+1, 9+6+3+1, 9+6+4+1, 9+6+5+1, 9+7+1, 9+7+2+1, 9+7+3+1, 9+7+4+1, 9+7+5+1, 9+7+6+1, 9+7+6+2+1, 9+7+6+3+1, 9+7+6+4+1, 9+7+6+5+1, 10+1, 10+2+1, 10+3+1, 10+4+1, 10+5+1, 10+6+1, 10+6+2+1, 10+6+3+1, 10+6+4+1, 10+6+5+1, 10+7+1, 10+7+2+1, 10+7+3+1, 10+7+4+1, 10+7+5+1, 10+7+6+1, 10+7+6+2+1, 10+7+6+3+1, 10+7+6+4+1, 10+7+6+5+1, 5+2+1, 5+3+1, 5+4+1, 6+1, 6+2+1, 6+3+1, 6+4+1, 6+5+1, 6+5+2+1, 6+5+3+1, 6+5+4+1, 7+1, 7+2+1, 7+3+1, 7+4+1, 7+5+1, 7+5+2+1, 7+5+3+1, 7+5+4+1, 7+6+1, 7+6+2+1, 7+6+3+1, 7+6+4+1, 7+6+5+1, 7+6+5+2+1, 7+6+5+3+1, 7+6+5+4+1, 8+1, 8+2+1, 8+3+1, 8+4+1, 8+5+1, 8+5+2+1, 8+5+3+1, 8+5+4+1, 8+6+1, 8+6+2+1, 8+6+3+1, 8+6+4+1, 8+6+5+1, 8+6+5+2+1, 8+6+5+3+1, 8+6+5+4+1, 9+1, 9+2+1, 9+3+1, 9+4+1, 9+5+1, 9+5+2+1, 9+5+3+1, 9+5+4+1, 9+6+1, 9+6+2+1, 9+6+3+1, 9+6+4+1, 9+6+5+1, 9+6+5+2+1, 9+6+5+3+1, 9+6+5+4+1, 9+7+1, 9+7+2+1, 9+7+3+1, 9+7+4+1, 9+7+5+1, 9+7+5+2+1, 9+7+5+3+1, 9+7+5+4+1, 9+7+6+1, 9+7+6+2+1, 9+7+6+3+1, 9+7+6+4+1, 9+7+6+5+1, 9+7+6+5+2+1, 9+7+6+5+3+1, 9+7+6+5+4+1, 10+1, 10+2+1, 10+3+1, 10+4+1, 10+5+1, 10+5+2+1, 10+5+3+1, 10+5+4+1, 10+6+1, 10+6+2+1, 10+6+3+1, 10+6+4+1, 10+6+5+1, 10+6+5+2+1, 10+6+5+3+1, 10+6+5+4+1, 10+7+1, 10+7+2+1, 10+7+3+1, 10+7+4+1, 10+7+5+1, 10+7+5+2+1, 10+7+5+3+1, 10+7+5+4+1, 10+7+6+1, 10+7+6+2+1, 10+7+6+3+1, 10+7+6+4+1, 10+7+6+5+1, 10+7+6+5+2+1, 10+7+6+5+3+1, 10+7+6+5+4+1, 11+10+1, 11+10+2+1, 11+10+3+1, 11+10+4+1, 11+10+5+1, 11+10+5+2+1, 11+10+5+3+1, 11+10+5+4+1, 11+10+6+1, 11+10+6+2+1, 11+10+6+3+1, 11+10+6+4+1, 11+10+6+5+1, 11+10+6+5+2+1, 11+10+6+5+3+1, 11+10+6+5+4+1, 11+10+7+1, 11+10+7+2+1, 11+10+7+3+1, 11+10+7+4+1, 11+10+7+5+1, 11+10+7+5+2+1, 11+10+7+5+3+1, 11+10+7+5+4+1, 11+10+7+6+1, 11+10+7+6+2+1, 11+10+7+6+3+1, 11+10+7+6+4+1, 11+10+7+6+5+1, 11+10+7+6+5+2+1, 11+10+7+6+5+3+1, 11+10+7+6+5+4+1, 12+10+1, 12+10+2+1, 12+10+3+1, 12+10+4+1, 12+10+5+1, 12+10+5+2+1, 12+10+5+3+1, 12+10+5+4+1, 12+10+6+1, 12+10+6+2+1, 12+10+6+3+1, 12+10+6+4+1, 12+10+6+5+1, 12+10+6+5+2+1, 12+10+6+5+3+1, 12+10+6+5+4+1, 12+10+7+1, 12+10+7+2+1, 12+10+7+3+1, 12+10+7+4+1, 12+10+7+5+1, 12+10+7+5+2+1, 12+10+7+5+3+1, 12+10+7+5+4+1, 12+10+7+6+1, 12+10+7+6+2+1, 12+10+7+6+3+1, 12+10+7+6+4+1, 12+10+7+6+5+1, 12+10+7+6+5+2+1, 12+10+7+6+5+3+1, 12+10+7+6+5+4+1, 13+1, 13+2+1, 13+3+1, 13+4+1, 13+5+1, 13+5+2+1, 13+5+3+1, 13+5+4+1, 13+6+1, 13+6+2+1, 13+6+3+1, 13+6+4+1, 13+6+5+1, 13+6+5+2+1, 13+6+5+3+1, 13+6+5+4+1, 13+7+1, 13+7+2+1, 13+7+3+1, 13+7+4+1, 13+7+5+1, 13+7+5+2+1, 13+7+5+3+1, 13+7+5+4+1, 13+7+6+1, 13+7+6+2+1, 13+7+6+3+1, 13+7+6+4+1, 13+7+6+5+1, 13+7+6+5+2+1, 13+7+6+5+3+1, 13+7+6+5+4+1, 14+10+1, 14+10+2+1, 14+10+3+1, 14+10+4+1, 14+10+5+1, 14+10+5+2+1, 14+10+5+3+1, 14+10+5+4+1, 14+10+6+1, 14+10+6+2+1, 14+10+6+3+1, 14+10+6+4+1, 14+10+6+5+1, 14+10+6+5+2+1, 14+10+6+5+3+1, 14+10+6+5+4+1, 14+10+7+1, 14+10+7+2+1, 14+10+7+3+1, 14+10+7+4+1, 14+10+7+5+1, 14+10+7+5+2+1, 14+10+7+5+3+1, 14+10+7+5+4+1, 14+10+7+6+1, 14+10+7+6+2+1, 14+10+7+6+3+1, 14+10+7+6+4+1, 14+10+7+6+5+1, 14+10+7+6+5+2+1, 14+10+7+6+5+3+1, 14+10+7+6+5+4+1, 14+11+10+1, 14+11+10+2+1, 14+11+10+3+1, 14+11+10+4+1, 14+11+10+5+1, 14+11+10+5+2+1, 14+11+10+5+3+1, 14+11+10+5+4+1, 14+11+10+6+1, 14+11+10+6+2+1, 14+11+10+6+3+1, 14+11+10+6+4+1, 14+11+10+6+5+1, 14+11+10+6+5+2+1, 14+11+10+6+5+3+1, 14+11+10+6+5+4+1, 14+11+10+7+1, 14+11+10+7+2+1, 14+11+10+7+3+1, 14+11+10+7+4+1, 14+11+10+7+5+1, 14+11+10+7+5+2+1, 14+11+10+7+5+3+1, 14+11+10+7+5+4+1, 14+11+10+7+6+1, 14+11+10+7+6+2+1, 14+11+10+7+6+3+1, 14+11+10+7+6+4+1, 14+11+10+7+6+5+1, 14+11+10+7+6+5+2+1, 14+11+10+7+6+5+3+1, 14+11+10+7+6+5+4+1, 14+12+10+1, 14+12+10+2+1, 14+12+10+3+1, 14+12+10+4+1, 14+12+10+5+1, 14+12+10+5+2+1, 14+12+10+5+3+1, 14+12+10+5+4+1, 14+12+10+6+1, 14+12+10+6+2+1, 14+12+10+6+3+1, 14+12+10+6+4+1, 14+12+10+6+5+1, 14+12+10+6+5+2+1, 14+12+10+6+5+3+1, 14+12+10+6+5+4+1, 14+12+10+7+1, 14+12+10+7+2+1, 14+12+10+7+3+1, 14+12+10+7+4+1, 14+12+10+7+5+1, 14+12+10+7+5+2+1, 14+12+10+7+5+3+1, 14+12+10+7+5+4+1, 14+12+10+7+6+1, 14+12+10+7+6+2+1, 14+12+10+7+6+3+1, 14+12+10+7+6+4+1, 14+12+10+7+6+5+1, 14+12+10+7+6+5+2+1, 14+12+10+7+6+5+3+1, 14+12+10+7+6+5+4+1, 15+10+1, 15+10+2+1, 15+10+3+1, 15+10+4+1, 15+10+5+1, 15+10+5+2+1, 15+10+5+3+1, 15+10+5+4+1, 15+10+6+1, 15+10+6+2+1, 15+10+6+3+1, 15+10+6+4+1, 15+10+6+5+1, 15+10+6+5+2+1, 15+10+6+5+3+1, 15+10+6+5+4+1, 15+10+7+1, 15+10+7+2+1, 15+10+7+3+1, 15+10+7+4+1, 15+10+7+5+1, 15+10+7+5+2+1, 15+10+7+5+3+1, 15+10+7+5+4+1, 15+10+7+6+1, 15+10+7+6+2+1, 15+10+7+6+3+1, 15+10+7+6+4+1, 15+10+7+6+5+1, 15+10+7+6+5+2+1, 15+10+7+6+5+3+1, 15+10+7+6+5+4+1, 15+11+10+1, 15+11+10+2+1, 15+11+10+3+1, 15+11+10+4+1, 15+11+10+5+1, 15+11+10+5+2+1, 15+11+10+5+3+1, 15+11+10+5+4+1, 15+11+10+6+1, 15+11+10+6+2+1, 15+11+10+6+3+1, 15+11+10+6+4+1, 15+11+10+6+5+1, 15+11+10+6+5+2+1, 15+11+10+6+5+3+1, 15+11+10+6+5+4+1, 15+11+10+7+1, 15+11+10+7+2+1, 15+11+10+7+3+1, 15+11+10+7+4+1, 15+11+10+7+5+1, 15+11+10+7+5+2+1, 15+11+10+7+5+3+1, 15+11+10+7+5+4+1, 15+11+10+7+6+1, 15+11+10+7+6+2+1, 15+11+10+7+6+3+1, 15+11+10+7+6+4+1, 15+11+10+7+6+5+1, 15+11+10+7+6+5+2+1, 15+11+10+7+6+5+3+1, 15+11+10+7+6+5+4+1, 15+12+10+1, 15+12+10+2+1, 15+12+10+3+1, 15+12+10+4+1, 15+12+10+5+1, 15+12+10+5+2+1, 15+12+10+5+3+1, 15+12+10+5+4+1, 15+12+10+6+1, 15+12+10+6+2+1, 15+12+10+6+3+1, 15+12+10+6+4+1, 15+12+10+6+5+1, 15+12+10+6+5+2+1, 15+12+10+6+5+3+1, 15+12+10+6+5+4+1, 15+12+10+7+1, 15+12+10+7+2+1, 15+12+10+7+3+1, 15+12+10+7+4+1, 15+12+10+7+5+1, 15+12+10+7+5+2+1, 15+12+10+7+5+3+1, 15+12+10+7+5+4+1, 15+12+10+7+6+1, 15+12+10+7+6+2+1, 15+12+10+7+6+3+1, 15+12+10+7+6+4+1, 15+12+10+7+6+5+1, 15+12+10+7+6+5+2+1, 15+12+10+7+6+5+3+1, 15+12+10+7+6+5+4+1, 16+10+1, 16+10+2+1, 16+10+3+1, 16+10+4+1, 16+10+5+1, 16+10+5+2+1, 16+10+5+3+1, 16+10+5+4+1, 16+10+6+1, 16+10+6+2+1, 16+10+6+3+1, 16+10+6+4+1, 16+10+6+5+1, 16+10+6+5+2+1, 16+10+6+5+3+1, 16+10+6+5+4+1, 16+10+7+1, 16+10+7+2+1, 16+10+7+3+1, 16+10+7+4+1, 16+10+7+5+1, 16+10+7+5+2+1, 16+10+7+5+3+1, 16+10+7+5+4+1, 16+10+7+6+1, 16+10+7+6+2+1, 16+10+7+6+3+1, 16+10+7+6+4+1, 16+10+7+6+5+1, 16+10+7+6+5+2+1, 16+10+7+6+5+3+1, 16+10+7+6+5+4+1, 16+11+10+1, 16+11+10+2+1, 16+11+10+3+1, 16+11+10+4+1, 16+11+10+5+1, 16+11+10+5+2+1, 16+11+10+5+3+1, 16+11+10+5+4+1, 16+11+10+6+1, 16+11+10+6+2+1, 16+11+10+6+3+1, 16+11+10+6+4+1, 16+11+10+6+5+1, 16+11+10+6+5+2+1, 16+11+10+6+5+3+1, 16+11+10+6+5+4+1, 16+11+10+7+1, 16+11+10+7+2+1, 16+11+10+7+3+1, 16+11+10+7+4+1, 16+11+10+7+5+1, 16+11+10+7+5+2+1, 16+11+10+7+5+3+1, 16+11+10+7+5+4+1, 16+11+10+7+6+1, 16+11+10+7+6+2+1, 16+11+10+7+6+3+1, 16+11+10+7+6+4+1, 16+11+10+7+6+5+1, 16+11+10+7+6+5+2+1, 16+11+10+7+6+5+3+1, 16+11+10+7+6+5+4+1, 16+12+10+1, 16+12+10+2+1, 16+12+10+3+1, 16+12+10+4+1, 16+12+10+5+1, 16+12+10+5+2+1, 16+12+10+5+3+1, 16+12+10+5+4+1, 16+12+10+6+1, 16+12+10+6+2+1, 16+12+10+6+3+1, 16+12+10+6+4+1, 16+12+10+6+5+1, 16+12+10+6+5+2+1, 16+12+10+6+5+3+1, 16+12+10+6+5+4+1, 16+12+10+7+1, 16+12+10+7+2+1, 16+12+10+7+3+1, 16+12+10+7+4+1, 16+12+10+7+5+1, 16+12+10+7+5+2+1, 16+12+10+7+5+3+1, 16+12+10+7+5+4+1, 16+12+10+7+6+1, 16+12+10+7+6+2+1, 16+12+10+7+6+3+1, 16+12+10+7+6+4+1, 16+12+10+7+6+5+1, 16+12+10+7+6+5+2+1, 16+12+10+7+6+5+3+1, 16+12+10+7+6+5+4+1, 17+10+1, 17+10+2+1, 17+10+3+1, 17+10+4+1, 17+10+5+1, 17+10+5+2+1, 17+10+5+3+1, 17+10+5+4+1, 17+10+6+1, 17+10+6+2+1, 17+10+6+3+1, 17+10+6+4+1, 17+10+6+5+1, 17+10+6+5+2+1, 17+10+6+5+3+1, 17+10+6+5+4+1, 17+10+7+1, 17+10+7+2+1, 17+10+7+3+1, 17+10+7+4+1, 17+10+7+5+1, 17+10+7+5+2+1, 17+10+7+5+3+1, 17+10+7+5+4+1, 17+10+7+6+1, 17+10+7+6+2+1, 17+10+7+6+3+1, 17+10+7+6+4+1, 17+10+7+6+5+1, 17+10+7+6+5+2+1, 17+10+7+6+5+3+1, 17+10+7+6+5+4+1, 17+11+10+1, 17+11+10+2+1, 17+11+10+3+1, 17+11+10+4+1, 17+11+10+5+1, 17+11+10+5+2+1, 17+11+10+5+3+1, 17+11+10+5+4+1, 17+11+10+6+1, 17+11+10+6+2+1, 17+11+10+6+3+1, 17+11+10+6+4+1, 17+11+10+6+5+1, 17+11+10+6+5+2+1, 17+11+10+6+5+3+1, 17+11+10+6+5+4+1, 17+11+10+7+1, 17+11+10+7+2+1, 17+11+10+7+3+1, 17+11+10+7+4+1, 17+11+10+7+5+1, 17+11+10+7+5+2+1, 17+11+10+7+5+3+1, 17+11+10+7+5+4+1, 17+11+10+7+6+1, 17+11+10+7+6+2+1, 17+11+10+7+6+3+1, 17+11+10+7+6+4+1, 17+11+10+7+6+5+1, 17+11+10+7+6+5+2+1, 17+11+10+7+6+5+3+1, 17+11+10+7+6+5+4+1, 17+12+10+1, 17+12+10+2+1, 17+12+10+3+1, 17+12+10+4+1, 17+12+10+5+1, 17+12+10+5+2+1, 17+12+10+5+3+1, 17+12+10+5+4+1, 17+12+10+6+1, 17+12+10+6+2+1, 17+12+10+6+3+1, 17+12+10+6+4+1, 17+12+10+6+5+1, 17+12+10+6+5+2+1, 17+12+10+6+5+3+1, 17+12+10+6+5+4+1, 17+12+10+7+1, 17+12+10+7+2+1, 17+12+10+7+3+1, 17+12+10+7+4+1, 17+12+10+7+5+1, 17+12+10+7+5+2+1, 17+12+10+7+5+3+1, 17+12+10+7+5+4+1, 17+12+10+7+6+1, 17+12+10+7+6+2+1, 17+12+10+7+6+3+1, 17+12+10+7+6+4+1, 17+12+10+7+6+5+1, 17+12+10+7+6+5+2+1, 17+12+10+7+6+5+3+1, 17+12+10+7+6+5+4+1, 18+10+1, 18+10+2+1, 18+10+3+1, 18+10+4+1, 18+10+5+1, 18+10+5+2+1, 18+10+5+3+1, 18+10+5+4+1, 18+10+6+1, 18+10+6+2+1, 18+10+6+3+1, 18+10+6+4+1, 18+10+6+5+1, 18+10+6+5+2+1, 18+10+6+5+3+1, 18+10+6+5+4+1, 18+10+7+1, 18+10+7+2+1, 18+10+7+3+1, 18+10+7+4+1, 18+10+7+5+1, 18+10+7+5+2+1, 18+10+7+5+3+1, 18+10+7+5+4+1, 18+10+7+6+1, 18+10+7+6+2+1, 18+10+7+6+3+1, 18+10+7+6+4+1, 18+10+7+6+5+1, 18+10+7+6+5+2+1, 18+10+7+6+5+3+1, 18+10+7+6+5+4+1, 18+11+10+1, 18+11+10+2+1, 18+11+10+3+1, 18+11+10+4+1, 18+11+10+5+1, 18+11+10+5+2+1, 18+11+10+5+3+1, 18+11+10+5+4+1, 18+11+10+6+1, 18+11+10+6+2+1, 18+11+10+6+3+1, 18+11+10+6+4+1, 18+11+10+6+5+1, 18+11+10+6+5+2+1, 18+11+10+6+5+3+1, 18+11+10+6+5+4+1, 18+11+10+7+1, 18+11+10+7+2+1, 18+11+10+7+3+1, 18+11+10+7+4+1, 18+11+10+7+5+1, 18+11+10+7+5+2+1, 18+11+10+7+5+3+1, 18+11+10+7+5+4+1, 18+11+10+7+6+1, 18+11+10+7+6+2+1, 18+11+10+7+6+3+1, 18+11+10+7+6+4+1, 18+11+10+7+6+5+1, 18+11+10+7+6+5+2+1, 18+11+10+7+6+5+3+1, 18+11+10+7+6+5+4+1, 18+12+10+1, 18+12+10+2+1, 18+12+10+3+1, 18+12+10+4+1, 18+12+10+5+1, 18+12+10+5+2+1, 18+12+10+5+3+1, 18+12+10+5+4+1, 18+12+10+6+1, 18+12+10+6+2+1, 18+12+10+6+3+1, 18+12+10+6+4+1, 18+12+10+6+5+1, 18+12+10+6+5+2+1, 18+12+10+6+5+3+1, 18+12+10+6+5+4+1, 18+12+10+7+1, 18+12+10+7+2+1, 18+12+10+7+3+1, 18+12+10+7+4+1, 18+12+10+7+5+1, 18+12+10+7+5+2+1, 18+12+10+7+5+3+1, 18+12+10+7+5+4+1, 18+12+10+7+6+1, 18+12+10+7+6+2+1, 18+12+10+7+6+3+1, 18+12+10+7+6+4+1, 18+12+10+7+6+5+1, 18+12+10+7+6+5+2+1, 18+12+10+7+6+5+3+1, or 18+12+10+7+6+5+4+1.

In the list above the numbers refer to the embodiments according to their numbering provided hereinabove whereas “+” indicates the dependency from another embodiment. The different individualized embodiments are separated by commas. In other words, “6+2+1” for example refers to embodiment 6) depending on embodiment 2), depending on embodiment 1), i.e. embodiment “6+2+1” corresponds to embodiment 1) further characterized by the features of the embodiments 2) and 6).

The term “amount” as used in the embodiments disclosed herein refers to “total amount” or “dose” or “total dose” or “dosage” or “total dosage”; especially said term refers to the total amount or total dose. It is understood that the terms “amount”, “total amount”, “dose”, “total dose”, “dosage”, “total dosage” as used herein are synonymous terms referring to the amount of active ingredient. Said terms may be used interchangeably within the context of the present invention and may be mutually replaced as appropriate and expedient.

The term “about” placed before a numerical value “X” in the current application refers to an interval extending from X minus 10% of X to X plus 10% of X; notably from X minus 5% of X to X plus 5% of X; especially said term refers to X. This applies also when the numerical value “X” in the current application itself is given in percent (%), e.g. if X is given as “about 0.5%”, this refers to the interval 0.45% to 0.55%; notably 0.475% to 0.525%; especially 0.5%.

For avoidance of doubt, it is understood that the amount of the active ingredient in “mg” in the embodiments disclosed herein refers to the total amount of said crystalline active ingredient administered to a patient within a 24-hour-period (e.g., 60 mg/day means that a total amount of 60 mg of the active ingredient is administered to a patient within 24 hours).

The term “mg/day” as used in the embodiments herein refers to milligrams per day and may be replaced with any of the following terms: “mg within a twenty-four-hour period”, “mg within twenty-four hours”, “mg per twenty-four hours”, “mg daily”, “mg per day” as appropriate and expedient.

The terms “subject(s)”, and likewise, “patient(s)” refers to mammal(s), especially human(s).

### EXPERIMENTAL PROCEDURES

### Abbreviations (as Used Herein)

DSC Differential scanning calorimetry
DCM Dichloromethane
DVS Dynamic vapor sorption
¹H-NMR Proton nuclear magnetic resonance
L Liter
min Minute(s)
mL Milliliter(s)
Ph. Eur. European Pharmacopeia
RH Relative humidity
RT Room temperature
XRPD X-ray powder diffraction

All solvents and reagents are used as obtained from commercial sources unless otherwise indicated. Temperatures are indicated in degrees Celsius (° C.). Unless otherwise indicated, the reactions take place at room temperature (RT).

X-Ray Powder Diffraction (XRPD)

XRPD method 1: X-ray powder diffraction patterns were collected on a Bruker D8 Advance X-ray diffractometer equipped with a Lynxeye detector operated in reflection mode (coupled two Theta/Theta). Typically, the Cu X-ray tube was run at of 40 kV/40 mA. A step size of 0.02° (2θ) and a step time of 76.8 seconds over a scanning range of 3-50° in 20 were applied. The divergence and the antiscatter slit were set to fixed 0.3°. Powders were slightly pressed into a silicon single crystal sample holder with depth of 0.5 mm and samples were rotated in their own plane during the measurement. Diffraction data are reported using Cu Kα (λ=1.5418 Å) radiation. The accuracy of the 2θ values as provided herein is in the range of +/−0.1-0.2° as it is generally the case for conventionally recorded X-ray powder diffraction patterns.

Dynamic Vapor Sorption (DVS)

Moisture sorption isotherm was collected on a multi sample instrument SPS-100n (Projekt Messtechnik, Ulm, Germany) operated in stepping mode at 25° C. The sample was allowed to equilibrate at 40% RH before starting a pre-defined humidity program 40-0-95-0-95-40% RH, steps of 5% RH and with a maximal equilibration time of 24 hours per step were applied. About 20 to 30 mg of each sample was used. The hygroscopic classification as disclosed herein is done according to the European Pharmacopeia Technical Guide (1999, page 86), that is, a material is classified as “not hygroscopic or “non-hygroscopic” when the increase in mass, as measured by the DVS method disclosed herein, is less than 0.2%; a material is classified as slightly hygroscopic, when the increase in mass is less than 2% and equal to or greater than 0.2% mass/mass; finally, a material is classified as hygroscopic, when the increase in mass is less than 15% and equal to or greater than 2% mass/mass. The mass change between 40% relative humidity and 80% relative humidity in the first adsorption scan was considered. Data shown are from the first upwards cycle.

Differential Scanning Calorimetry (DSC)

DSC data were collected on a Mettler Toledo STARe System (DSC822e module, measuring cell with ceramic sensor and STAR software version 9.20) equipped with a 34-position auto-sampler. The instrument was calibrated for energy and temperature using certified indium. Typically, 1-5 mg of each sample (5-6 mg for Reference Example 1), in an automatically pierced aluminum pan, was heated at 10° C. min⁻¹ (20° C. min⁻¹ for Reference Example 1), unless stated otherwise, from −20° C. to 280° C. A nitrogen purge at 20 mL min⁻¹ was maintained over the sample. Peak temperatures are reported for melting points.

Bulk Density and Tapped Density

Bulk and tapped density were measured on a Densi-tap tapping device from Matec using a 10 mL glass cylinder and using powder without any processing. For tapped density the value is reported after 3750 tappings.

### EXAMPLES

ACT-539313 may be prepared according to the procedure given in Example 27 of WO2013068935. Said procedure furnishes ACT-539313 in amorphous state.

### Example 1

0.05 mL of 1-propanol is added to 50 mg of ACT-539313 in amorphous state in a standard glass 4-mL-vial. After 1 day of closed storage at room temperature a solid material is isolated showing an XRPD diagram of ACT-539313 in crystalline form 1 (FIG. 1).

### Example 2

0.05 mL of ethanol is added to 50 mg of ACT-539313 in amorphous state in a standard glass 4-mL-vial. After 1 day of closed storage at room temperature a solid material is isolated showing an XRPD diagram of ACT-539313 in crystalline form 1 (FIG. 1).

### Example 3

0.3 mL of 2-propanol is added to 300 mg of ACT-539313 in amorphous state in a standard glass 4-mL-vial. After 1 day of closed storage at room temperature a solid material is isolated showing an XRPD diagram of ACT-539313 in crystalline form 1 (FIG. 1).

TABLE 1
Characterization data for ACT-539313 in crystalline form 1

| Technique | Data Summary | Remarks |
|---|---|---|
| XRPD | Crystalline | FIG. 1 |
| ¹H-NMR | Consistent | |
| DSC | Melt endotherm with melting point at | FIG. 3 |
| | T = 117.6 ± 2° C. | |
| Moisture sorption | ACT-539313 in crystalline form 1 is not | FIG. 4 |
| at 25° C. (DVS) | hygroscopic according to Ph. Eur. | |
| | Material picks up about 0.1% | |
| | of moisture in the range 0 | |
| | to 95% RH at 25° C. | |

### Example 4

About 450 g of ACT-539313 were dissolved in 4 L 2-propanol at 70° C. About 1 L of solvent was distilled off, the remaining solution was transferred into a 4 L vessel and stirred at 50° C. while seeded with about 113 g of ACT-539313 in crystalline form 1. Seeding material can be prepared according to the procedures described in Examples 1 to 3 after appropriate scale up. The solution was then stirred for about 1 h at 5000 while solid product precipitated. Heating was switched off, the suspension was left to cool down to room temperature and stirred overnight, resulting in 506 g (90%) of ACT-539313 in crystalline form 1. The crystalline product had a bulk density of 0.31 g/mL and a tapped density of 0.46 g/mL as measured according to the method described herein.

### Example 5

Certain pharmaceutical compositions (25, 30, 50, 100 mg API) suitable for administration in humans are shown in Table 2.

TABLE 2

Pharmacopeial

name (Ph. Eur.)/

| Material Name | Function | Composition per unit [%] | | | | Quantity per unit [mg] | | | |
|---|---|---|---|---|---|---|---|---|---|
| —/ACT-539313 | API | 23.81 | 11.90 | 7.14 | 5.95 | 100.00 | 50.00 | 30.00 | 25.00 |
| Silica Colloidal | Lubricant | 0.95 | 0.48 | 0.29 | 0.24 | 4.00 | 2.00 | 1.20 | 1.00 |
| Hydrated/Syloid | | | | | | | | | |
| 244 FP | | | | | | | | | |
| Microcrystalline | Filler/Binder | 41.74 | 49.28 | 51.54 | 53.04 | 175.31 | 206.96 | 216.48 | 222.78 |
| Cellulose/Avicel | | | | | | | | | |
| PH 101 | | | | | | | | | |
| Mannitol (EP)/ | Disintegrant | 26.83 | 31.68 | 34.36 | 34.10 | 112.69 | 133.04 | 144.32 | 143.22 |
| Parteck M 200 | | | | | | | | | |
| Emprove | | | | | | | | | |
| Crospovidone (EP)/ | Binder | 4.76 | 4.76 | 4.76 | 4.76 | 20.00 | 20.00 | 20.00 | 20.00 |
| Kollidon CL | | | | | | | | | |
| Sodium Stearyl | Lubricant | 0.48 | 0.48 | 0.48 | 0.48 | 2.00 | 2.00 | 2.00 | 2.00 |
| Fumarate/Pruv | | | | | | | | | |
| Total inner phase | | 98.57 | 98.57 | 98.57 | 98.57 | 414.00 | 414.00 | 414.00 | 414.00 |
| Silica Colloidal | Lubricant | 0.95 | 0.95 | 0.95 | 0.95 | 4.00 | 4.00 | 4.00 | 4.00 |
| Hydrated/Syloid | | | | | | | | | |
| 244 FP | | | | | | | | | |
| Sodium Stearyl | Lubricant | 0.48 | 0.48 | 0.48 | 0.48 | 2.00 | 2.00 | 2.00 | 2.00 |
| Fumarate/Pruv | | | | | | | | | |
| Total final blend | | 100.0 | 100.0 | 100.0 | 100.0 | 420.00 | 420.0 | 420.0 | 420.0 |

Capsule manufacturing method: ACT-539313, microcrystalline cellulose, mannitol, crosspovidone, and sodium stearyl fumarate were blended in an RRM-mixer for 5 min at 37 rpm; sieved (0.8 mm screen); and further blended for 5 min at 37 rpm. The resulting inner phase blend was dry granulated (7.5 kN/5 rpm) and further blended for 10 min at 37 rpm. Sieved (0.8 mm screen) silica colloidal hydrated was added, the blend was further mixed for 5 min at 37 rpm, followed by addition of sieved (0.8 mm screen) sodium stearyl fumarate and final blending for 5 min at 37 rpm. The resulting final blend was encapsulated in hard-gelatine capsules size 0 using Modu-C LS equipment. Tablet manufacturing method: surprisingly, the final blend (25 mg, 50 mg, and 100 mg API) used for the manufacturing of capsules could be successfully compressed into round and/or oblong tablets of different sizes (e.g. 10, 12, 13, and 17 mm) without substantial modifications of the composition.

### REFERENCE EXAMPLES

### Reference Example 1

50 mL of DCM were added to 10 g of ACT-539313 in a 2 L round bottom flask and the solvent was removed on a rotary evaporator operated at 55° C. and 5 mbar until complete dryness of the solid residue. The XRPD diagram of the solid material is shown in FIG. 2. The resulting amorphous ACT-539313 had a bulk density of 0.06 g/mL and a tapped density of 0.13 g/mL as measured according to the method described herein.

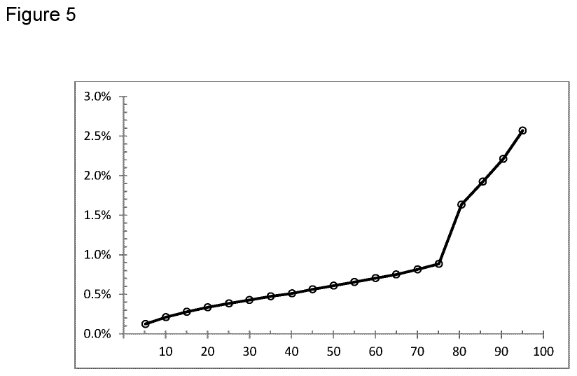

TABLE 3
Characterization data for ACT-539313 in amorphous state

| Technique | Data Summary | Remarks |
|---|---|---|
| XRPD | Amorphous | FIG. 2 |
| ¹H-NMR | Consistent | |
| DSC | Glass transition temperature | |
| | (T_{g}) = 60 ± 10° C. | |
| Moisture sorption | ACT-539313 in amorphous state is | FIG. 5 |
| at 25° C. (DVS) | slightly hygroscopic according to | |
| | Ph. Eur. | |
| | Material picks up about | |
| | 2.6% of moisture in the range 0 | |
| | to 95% RH at 25° C. | |

### Reference Example 2

19 vials, each containing about 10 mg of ACT-539313, were prepared by evaporation in the vial from a DCM stock solution, resulting in a transparent (i.e. amorphous) film in the vial. In a systematic way, the influence of 2-propanol added volume (0.01, 0.02, 0.04, 0.08, 0.16, 0.30, and 0.5 mL) was investigated, as well as 0.02 mL added volume of alternative organic solvents commonly used in crystallization (methanol, ethanol, ethyl acetate, water, tert-butyl methyl ether, dioxane, acetone, methyl isobutyl ketone, tetrahydrofuran, toluene, and dimethyl sulfoxide). Only within 30 min samples from 2-propanol at minimal volumes in the range from 0.01 mL to 0.16 mL showed signs of crystallization (as observed under the light stereomicroscope with crossed polars). In contrast thereto, ethanol showed first signs of crystallization only 1.25 h later. At about 6 h no other solvent showed signs of crystallization and only when visually checked after 3 days crystallization was observed from water, tert-butyl methyl ether and methyl isobutyl ketone.

Crystalline form 1 of ACT-539313 as described herein is less hygroscopic than the amorphous ACT-539313 (cf. FIGS. 4 and 5; and Tables 1 and 3). Further, said crystalline form melts at about 118° C. as compared to the amorphous material which has a glass transition temperature in the range 50-70° C. Also, the bulk density of the amorphous ACT-539313 (0.06 g/mL) is significantly lower than the bulk density of ACT-539313 in crystalline form 1 (0.31 g/ml). Materials with lower density are fluffier and thereby more difficult to handle (e.g. require larger processing vessels). Based on the measured tapped and bulk densities, the flowability of the bulk material, as expressed by its Hausner ratio (HR) (ratio between tapped and bulk density), can be calculated. HR for the crystalline form 1 of ACT-539313 (Example 4) is 1.47, suggesting a type of powder flow for this material that may be classified as “very poor” (HR from 1.46 to 1.59). However, HR of the amorphous material (Reference Example 1) is about 150% higher, namely 2.26, suggesting a type of flow that is classified as “very, very poor” (HR>1.60).

1. A crystalline form of (4-Methyl-2-[1,2,3]triazol-2-yl-phenyl)-[(R)-3-(3-[1,2,3]triazol-2-yl-benzyl)-morpholin-4-yl]-methanone, characterized by the presence of peaks in the X-ray powder diffraction diagram at the following angles of refraction 2θ: 10.5°, 14.2°, and 18.4°.

2. The crystalline form according to claim 1, characterized by the presence of peaks in the X-ray powder diffraction diagram at the following angles of refraction 2θ: 7.8°, 10.5°, 14.2°, 18.4°, and 21.80.

3. The crystalline form according to claim 1, characterized by the presence of peaks in the X-ray powder diffraction diagram at the following angles of refraction 2θ: 7.8°, 10.5°, 12.5°, 13.7°, 14.2°, 14.60, 18.40, 21.40, 21.80, and 25.10.

4. The crystalline form according to claim 1, which essentially shows the X-ray powder diffraction pattern as depicted in FIG. 1.

5. The crystalline form according to claim 1, characterized by a melting point of about 117.6° C. as determined by differential scanning calorimetry.

6. The crystalline form according to claim 1, obtainable by a process comprising
a. dissolving (4-Methyl-2-[1,2,3]triazol-2-yl-phenyl)-[(R)-3-(3-[1,2,3]triazol-2-yl-benzyl)-morpholin-4-yl]-methanone in a solvent, said solvent comprising one or more lower alcohols;
b. awaiting formation of a solid product; and
c. isolating the solid product.

7. A process for making the crystalline form according to claim 1, said process comprising recrystallization of (4-Methyl-2-[1,2,3]triazol-2-yl-phenyl)-[(R)-3-(3-[1,2,3]triazol-2-yl-benzyl)-morpholin-4-yl]-methanone in a solvent, said solvent comprising one or more lower alcohols.

8. A pharmaceutical composition comprising as active ingredient the crystalline form according to claim 1, and at least one pharmaceutically acceptable carrier.

9. A solid dosage form, wherein the solid dosage form comprises a crystalline form of (4-Methyl-2-[1,2,3]triazol-2-yl-phenyl)-[(R)-3-(3-[1,2,3]triazol-2-yl-benzyl)-morpholin-4-yl]-methanone, wherein the crystalline form is characterized by the presence of peaks in the X-ray powder diffraction diagram at the following angles of refraction 2θ: 10.5°, 14.2°, and 18.4°.

10. The pharmaceutical composition according to claim 8, in form of a tablet for oral use, said composition comprising
from about 3% w/w to about 40% w/w of said active ingredient;
from about 30% w/w to about 70% w/w microcrystalline cellulose;
from about 15% w/w to about 60% w/w mannitol;
from about 3% w/w to about 10% w/w crosspovidone;
from about 0.2% w/w to about 4% w/w sodium stearyl fumarate; and
from about 0.5% w/w to about 5% w/w silica colloidal hydrated.

11. The pharmaceutical composition according to claim 8, in form of a tablet for oral use, said composition comprising
from 22% to 25% w/w of said active ingredient;
from 39% to 44% w/w microcrystalline cellulose;
from 24% to 30% w/w mannitol;
from 4% to 6% w/w crosspovidone;
from 0.5% to 1.5% w/w sodium stearyl fumarate; and
from 1.5% to 2.5% w/w silica colloidal hydrated;
or
from 10% to 13% w/w of said active ingredient;
from 47% to 52% w/w microcrystalline cellulose;
from 29% to 34% w/w mannitol;
from 4% to 6% w/w crosspovidone;
from 0.5% to 1.5% w/w sodium stearyl fumarate; and
from 1% to 2% w/w silica colloidal hydrated;
or
from 6% to 8% w/w of said active ingredient;
from 48% to 54% w/w microcrystalline cellulose;
from 31% to 35% w/w mannitol;
from 4% to 6% w/w crosspovidone;
from 0.5% to 1.5% w/w sodium stearyl fumarate; and
from 1% to 2% w/w silica colloidal hydrated;
or
from 5% to 7% w/w of said active ingredient;
from 50% to 55% w/w microcrystalline cellulose;
from 30% to 35% w/w mannitol;
from 4% to 6% w/w crosspovidone;
from 0.5% to 1.5% w/w sodium stearyl fumarate; and
from 0.5% to 2% w/w silica colloidal hydrated.

12. (canceled)

13. A method for the prevention and/or treatment of anxiety disorders, addiction disorders, mood disorders, appetite disorders, cognitive dysfunctions, or sleep disorders in a patient in need thereof, wherein the method comprises administering to the patient a crystalline form of (4-Methyl-2-[1,2,3]triazol-2-yl-phenyl)-[(R)-3-(3-[1,2,3]triazol-2-yl-benzyl)-morpholin-4-yl]-methanone, wherein the crystalline form is characterized by the presence of peaks in the X-ray powder diffraction diagram at the following angles of refraction 2θ: 10.50, 14.20, and 18.40.

14. The method according to claim 13, wherein the method is for the prevention and/or treatment of an eating disorder selected from a group comprising Binge-Eating Disorder (BED); Bulimia Nervosa (BN); Anorexia Nervosa (AN); Pica; Other Specified Feeding and Eating Disorders (OSFED); Unspecified Feeding or Eating Disorder (UFED); Eating Disorder Not Otherwise Specified (EDNOS); Compulsive Overeating (CO); Loss of Control (LOC) Eating; and hyperphagia and/or binge-eating, associated with Prader-Willi Syndrome (PWS).

15. The method according to claim 14, wherein the method is for the prevention and/or treatment of Binge-Eating Disorder (BED).

16. The pharmaceutical composition according to claim 8, wherein the crystalline form is characterized by the presence of peaks in the X-ray powder diffraction diagram at the following angles of refraction 2θ: 7.8°, 10.5°, 12.5°, 13.7°, 14.2°, 14.6°, 18.4°, 21.4°, 21.8°, and 25.1°.

17. The solid dosage form according to claim 9, wherein the crystalline form is characterized by the presence of peaks in the X-ray powder diffraction diagram at the following angles of refraction 2θ: 7.80, 10.50, 12.50, 13.70, 14.20, 14.60, 18.40, 21.40, 21.80, and 25.1°.

18. The method according to claim 14, wherein the crystalline form is characterized by the presence of peaks in the X-ray powder diffraction diagram at the following angles of refraction 2θ: 7.8°, 10.50, 12.50, 13.70, 14.20, 14.60, 18.40, 21.40, 21.80, and 25.1°.

19. The method according to claim 15, wherein the crystalline form is characterized by the presence of peaks in the X-ray powder diffraction diagram at the following angles of refraction 2θ: 7.8°, 10.50, 12.50, 13.70, 14.20, 14.60, 18.40, 21.40, 21.80, and 25.1°.

20. A method for preparing a solid pharmaceutical composition comprising (4-Methyl-2-[1,2,3]triazol-2-yl-phenyl)-[(R)-3-(3-[1,2,3]triazol-2-yl-benzyl)-morpholin-4-yl]-methanone and at least one pharmaceutically acceptable carrier material, wherein the method comprises mixing a crystalline form of (4-Methyl-2-[1,2,3]triazol-2-yl-phenyl)-[(R)-3-(3-[1,2,3]triazol-2-yl-benzyl)-morpholin-4-yl]-methanone with the at least one pharmaceutically acceptable carrier material, and wherein the crystalline form is characterized by the presence of peaks in the X-ray powder diffraction diagram at the following angles of refraction 2θ: 10.5°, 14.2°, and 18.4°.

21. The method according to claim 20, wherein the crystalline form is characterized by the presence of peaks in the X-ray powder diffraction diagram at the following angles of refraction 2θ: 7.8°, 10.50, 12.50, 13.70, 14.20, 14.60, 18.40, 21.40, 21.80, and 25.1°.
